(19) United States
(12) Patent Application Publication
(10) Pub. No.: US 2013/0149265 A1
(43) Pub. Date: Jun. 13, 2013

(54) Sunscreen

(71) Applicant: Jennifer Lawandus, Lancaster, NY (US)

(72) Inventor: Jennifer Lawandus, Lancaster, NY (US)

(21) Appl. No.: 13/709,587

(22) Filed: Dec. 10, 2012

Related U.S. Application Data
(60) Provisional application No. 61/568,775, filed on Dec. 9, 2011.

(51) Int. Cl. A61K 8/97; A61Q 17/04

(52) U.S. Cl. CPC A61K 8/97; A61Q 17/04
USPC 424/59

(57) ABSTRACT

A sunscreen composition which has an aloe vera concentrate and a distilled water mixture which is heated creating a first mixture. A coconut oil and a polyglycerol mixture are blended with zinc oxide and beeswax before heated to create a third mixture. The first mixture is blended with the third mixture to create a lotion texture. A titanium dioxide and a pigment blend are mixed to create a second mixture which is poured into the combined first and third mixtures. This mixture is then blended to create the lotion texture. Paraben-DU is lastly added to the mixture and blended until the lotion texture is created again. This final mixture is refrigerated and then again blended to create the lotion texture. Lastly, the mixture is placed in direct sunlight and any visible lumps are crushed before storage.

### CROSS REFERENCE TO RELATED APPLICATION

This application claims the benefit of and takes priority from U.S. Provisional Patent application Ser. No. 61/568,775, filed on Dec. 9, 2011, the contents of which are herein incorporated by reference.

### FIELD OF THE INVENTION

The present invention relates to cream compositions protecting skin again UVA and/or UVB rays.

### BACKGROUND OF THE INVENTION

A variety of protective creams is known and may include sunscreen variations containing a diester of butyl alcohol and adipic acid. Other known sunscreens contain pure, micronized, and/or encapsulated insert minerals such as, titanium dioxide and zinc oxide for the purpose of protecting skin against UVA and/or UVB rays.

The sunscreens containing dibutyl adipate or any variation of a diester of butyl alcohol and adipic acid cause acrylic nails and/or nail polish to break down and become tacky. The known sunscreens containing pure, micronized, and/or encapsulated inert minerals may also cause a white visible film residue on human skin after application.

### SUMMARY OF THE INVENTION

The instant invention, as illustrated herein, is clearly not anticipated, rendered obvious, or even present in any of the prior art mechanisms, either alone or in any combination thereof. It is therefore an object of this system and method to provide a sunscreen formulation that does not adversely affect acrylic nails and/or nail polish.

It is a further object of this invention to provide a sunscreen that makes the skin appear tan while protecting the skin from UVA and/or UVB rays in order to mask the white film produced by pure, micronized, and/or encapsulated inert minerals such as, titanium dioxide and zinc oxide when applied to the skin.

The subject invention results from the realization that the problems of acrylic nails and/or nail polish being broken down and becoming tacky and white film residue are addressed in a sunscreen free of dibutyl adipate or any variation of diester of butyl alcohol and adipic acid and a sunscreen containing a pigment blend to mask the white film produced by pure, micronized, and/or encapsulated inert minerals and to darken the consumer's skin temporarily when wearing sunscreen.

The invention features the sunscreen composition comprising an aloe vera and green mixture, a titanium dioxide and pigment mixture, and a coconut oil, polyglycerol oleate, zinc oxide, and beeswax mixture. It has been found that altering the component amounts of these mixtures produces various thicknesses of the cream, a non-waterproof cream, larger or smaller batches of the sunscreen, as well as increasing or decreasing the SPF.

There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description. The invention is capable of other embodiments and of being practiced and carried out in various ways. Also, it is to be understood that the phraseology and terminology employed herein are for the purpose of the description and should not be regarded as limiting.

These together with other objects of the invention, along with the various features of novelty, which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention. Other features and advantages of the present invention will become apparent from the following description of the preferred embodiment(s), taken in conjunction with the accompanying drawings, which illustrate, by way of example, the principles of the invention.

There has thus been outlined, rather broadly, the more important features of the versatile sunscreen formulation embodiments in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The foregoing has outlined the more pertinent and important features of the present invention in order that the detailed description of the invention that follows may be better understood, and the present contributions to the art may be more fully appreciated. It is of course not possible to describe every conceivable combination of components and/or methodologies, but one of ordinary skill in the art may recognize that many further combinations or permutations are possible. Accordingly, the novel architecture described below is intended to embrace all such alterations, modifications, and variations that fall within the spirit and scope of the appended claims.

### DETAILED DESCRIPTION OF THE INVENTION

The detailed description set forth below is intended as a description of presently preferred embodiments of the invention and does not represent the only forms in which the present invention may be construed and/or utilized. The description sets forth the functions and the sequence of steps for producing the invention. However, it is to be understood that the same or equivalent functions and sequences may be accomplished by different embodiments also intended to be encompassed within the spirit and scope of the invention.

The unique characteristic of the present invention is the ability to protect skin from UVA and/or UVB rays while avoiding white residue on the skin and without breaking down the texture of acrylic nails and/or nail polish.

The composition of the present invention includes a first mixture of aloe vera and distilled water or green tea made with distilled water refrigerated in a second beaker. These components are thought to contain cancer cell fighting characteristics and reduce the damage to healthy cells. A second mixture in a first bowl for the composition of this invention is a blend of titanium dioxide and pigment mixed at room temperature until pigment is evenly distributed. The last mixture for the composition of this invention is a blend of coconut oil and polyglycerol oleate in a first beaker. Zinc oxide is slowly added to this last mixture and lastly beeswax is added. It is known in the art that zinc oxide and titanium dioxide are important elements due to their capacity in protecting skin against UVB rays which produce sunburns.

The last mixture is heated at no more than 140 degrees Fahrenheit until the beeswax turns to liquid. Substantially 30 seconds before this last mixture has completely melted the first mixture should be heated in a microwave with a RF output frequency of 2450 MHz for substantially 30 seconds. Both the last mixture and the first mixture are removed from the heat and tested for a temperature of 135 degrees Fahrenheit. The last mixture is poured into the first mixture when the desired temperature has been reached. The last and first mixtures are blended together in a second bowl to resemble a lotion texture. The contents of the second bowl should be further blended at a more rapid speed until the mixture begins to separate and then blended once more until resembles the texture of lotion.

Substantially one quarter of the second mixture is added to the blended mixture in the second bowl. The remaining second mixture is slowly added to the second bowl while being blended. Paraben-DU is then slowly added to this mixture in the second bowl. At a rapid speed the contents of the second bowl are blended until the mixture begins to separate. Then the mixture is blended once more until resembles a lotion texture.

The mixture in the second glass bowl is refrigerated for substantially 15 minutes. After refrigeration the mixture is once again blended together until resemble texture of a lotion. The contents are further blended at a more rapid speed until the mixture begins to separate. The mixture is then blended once more until resembles a lotion texture. The mixture in the second bowl is then placed in direct sunlight and any lumps or particles visible are crushed. Once no more lumps are visible the mixture is blended a final time until resembles lotion texture.

In another embodiment or variation, the ingredients of the sunscreen can be increased or decreased in measurement to create a larger or smaller batch of sunscreen. In an additional embodiment the measurement of beeswax can be increased while maintaining the same percentage of accompanied ingredients to create a thicker textured sunscreen. A thinner variation of the sunscreen comprises either increasing the measurement of distilled water or green tea made with distilled water while maintaining the same percentage of accompanied ingredients or decreasing the measurement of beeswax while maintaining the same percentage of accompanied ingredients. To alter the SPF of the sunscreen the measurement of pure, micronized, and/or encapsulated inert minerals such as, titanium dioxide and zinc oxide can be either increased or decreased. In such an embodiment, either increasing or decreasing the measurement of beeswax while maintaining the same percentage of accompanied ingredients can maintain the texture of the lotion. In a final embodiment, removing the polyglyceryl oleate from the ingredients creates a non-waterproof sunscreen. In such an embodiment, the texture of the lotion is maintained by either increasing the measurement of distilled water or green tea or decreasing the measurement of beeswax both while maintaining the same percentage of accompanied ingredients.

In a further embodiment, the following increased of ingredients to the formulation may be utilized: One Teaspoon (1 TSP) Lecithin (mixing a coconut oil, lechithin, and polyglycerol oleate to create a third mixture); One eighth Teaspoon (⅛ TSP) Sodium Tetraborate (mixing an aloe vera and sodium tetraborate with a distilled water to create a first mixture).

In a further embodiment, the Aloe Vera 10×Concentrate may be decreased to One Tablespoon (1 TBSP). Regarding active ingredients, the sunscreen solutions herein could be formulated with the following active ingredients: Tinosorb S, Tinosorb M, Mexoryl SX

What is claimed is:

1. A sunscreen composition comprising:
an aloe vera concentrate and a distilled water mixture;
a titanium dioxide and a pigment blend;
a coconut oil, a polyglycerol oleate, a zinc oxide, and a beeswax mixture; and
a paraben-DU.

2. The sunscreen composition of claim 1 wherein:
about 5.25% to about 7.25% of a total amount of the composition is the aloe vera; and
about 24% to about 26% of the total amount of the composition is the distilled water.

3. The sunscreen composition of claim 1 wherein:
about 4.2% to about 6.2% of the total amount of the composition is the titanium dioxide; and
about 0.42% to about 0.62% of the total amount of the composition is the pigment blend.

4. The sunscreen composition of claim 1 wherein:
about 11.5% to about 13.5% of the total amount of the composition is the coconut oil;
about 24% to about 26% of the total amount of the composition is the polyglycerol oleate;
about 15% to about 17% of the total amount of the composition is the zinc oxide; and
about 7.3% to about 9.3% of the total amount of the composition is the beeswax mixture.

5. The sunscreen composition of claim 1 wherein the Paraben-DU is about 0.42% to about 0.62% of the total amount of the composition.

6. The sunscreen composition of claim 1 further comprising a quantity of one teaspoon lecithin and one eighth teaspoon sodium tetraborate.

7. The sunscreen composition of claim 6 wherein the lecithin comprises a mixture of a coconut oil, lechithin, and polyglycerol oleate.

8. The sunscreen composition of claim 6 wherein the sodium tetraborate comprises mixture of an aloe vera and sodium tetraborate with a quantity distilled water.

9. The sunscreen composition of claim 1 wherein a set of active ingredients may be selected from the group consisting of tinosorb s, tinosorb m, and mexoryl sx.

10. A method of producing an improved sunscreen comprising:
mixing a quantity of aloe vera with a quantity of distilled water to create a first mixture;
mixing a quantity of titanium dioxide with a quantity of pigment blend to create a second mixture;
mixing a quantity of coconut oil and a quantity of polyglycerol oleate to create a third mixture;
pouring a quantity of zinc oxide into the third mixture;
pouring a quantity of beeswax into the third mixture;
heating the third mixture until the third mixture liquid;
heating the first mixture;
removing the first and third mixture from heat;
pouring the third mixture into the first mixture;
blending the first and third mixture until lotion texture;
pouring the second mixture into a combined first and third mixture;
blending the second and the combined first and third mixture until lotion texture;
adding a quantity of Paraben-DU to a combined first, second, and third mixture to create a final mixture;
blending the final mixture until lotion texture;
refrigerating the final mixture;
blending the final mixture;
placing the final mixture in direct sunlight;
crushing any visible lumps; and
storing the final mixture.

11. The method of producing an improved sunscreen of claim 10 wherein about 5.25% to about 7.25% of a total amount of the composition is the aloe vera and about 24% to about 26% of the total amount of the composition is the distilled water.

12. The method of producing an improved sunscreen of claim 10 wherein about 4.2% to about 6.2% of the total amount of the composition is the titanium dioxide and about 0.42% to about 0.62% of the total amount of the composition is the pigment blend.

13. The method of producing an improved sunscreen of claim 10 wherein about 11.5% to about 13.5% of the total amount of the composition is the coconut oil, about 24% to about 26% of the total amount of the composition is the polyglycerol oleate, about 15% to about 17% of the total amount of the composition is the zinc oxide, and about 7.3% to about 9.3% of the total amount of the composition is the beeswax mixture.

14. The method of producing an improved sunscreen of claim 10 wherein the Paraben-DU is about 0.42% to about 0.62% of the total amount of the composition.
